SEQUENCE LISTING (15 sequences)

SEQ ID NO: 1 - DNA, 563 bp, Artificial Sequence
  Synthesized using sequential PCR techniques
ggatatccaccatggacttcgggttgagcttggttttccttgtccttactttaaaaggtgtccagtgtgatagccacgctaagcggcaccacggatataagcggaagttccacgagaagcaccactcgcacagaggatactctggtggcggtggctcgggcggaggtgggtcgggtggcggcggatccgacgtgaagcttgtggagtctgggggaggcttagtgaaccctggagggtccctgaaactctcctgtgcagcctctggattcactttcagtagctataccatgtcttgggttcgccagactccggagaagaggctggagtgggtcgcatccattagtagtggtggtacttacacctactatccagacagtgtgaagggccgattcaccatctccagagacaatgccaagaacaccctgtacctgcaaatgaccagtctgaagtctgaggacacagccatgtattactgttcaagagatgacggctcctacggctcctattactatgctatggactactggggtcaaggaacctcagtcaccgtctcttcagctagc

SEQ ID NO: 2 - protein, 24 aa, Artificial Sequence
  Synthesized using sequential PCR techniques
DSHAKRHHGYKRKFHEKHHSHRGY

SEQ ID NO: 3 - protein, 16 aa, Artificial Sequence
  Synthesized using sequential PCR techniques
SGGGGSGGGGSGGGGS

SEQ ID NO: 4 - protein, 125 aa, Artificial Sequence
  Synthesized using sequential PCR techniques
DVKLVESGGGLVNPGGSLKLSCAASGFTFSSYTMSWVRQTPEKRLEWVASISSGGTYTYYPDSVKGRFTISRDNAKNTLYLQMTSLKSEDTAMYYCSRDDGSYGSYYYAMDYWGQGTSVTVSSAS

SEQ ID NO: 5 - DNA, 533 bp, Artificial Sequence
  Synthesized using squential PCR techniques
ggatatccaccatggacttcgggttgagcttggttttccttgtccttactttaaaaggtgtccagtgtaagcggctgtttaaggagctcaagttcagcctgcgcaagtactctggtggcggtggctcgggcggaggtgggtcgggtggcggcggatccgacgtgaagcttgtggagtctgggggaggcttagtgaaccctggagggtccctgaaactctcctgtgcagcctctggattcactttcagtagctataccatgtcttgggttcgccagactccggagaagaggctggagtgggtcgcatccattagtagtggtggtacttacacctactatccagacagtgtgaagggccgattcaccatctccagagacaatgccaagaacaccctgtacctgcaaatgaccagtctgaagtctgaggacacagccatgtattactgttcaagagatgacggctcctacggctcctattactatgctatggactactggggtcaaggaacctcagtcaccgtctcttcagctagc

SEQ ID NO: 6 - protein, 14 aa, Artificial Sequence
  Synthesized using squential PCR techniques
KRLFKELKFSLRKY

SEQ ID NO: 7 - protein, 16 aa, Artificial Sequence
  Synthesized using squential PCR techniques
SGGGGSGGGGSGGGGS

SEQ ID NO: 8 - protein, 125 aa, Artificial Sequence; identical to SEQ ID NO: 4
  Synthesized using squential PCR techniques

SEQ ID NO: 9 - DNA, 89 bp, Artificial Sequence
  Primer 986
caccactcgcacagaggatactctggtggcggtggctcgggcggaggtgggtcgggtggcggcggatccgacgtgaagcttgtggagtc

SEQ ID NO: 10 - DNA, 84 bp, Artificial Sequence
  Primer 987
ggtgtccagtgtgatagccacgctaagcggcaccacggatataagcggaagttccacgagaagcaccactcgcacagaggatac

SEQ ID NO: 11 - DNA, 74 bp, Artificial Sequence
  Primer 988
gatatccaccatggacttcgggttgagcttggttttccttgtccttactttaaaaggtgtccagtgtgatagcc

SEQ ID NO: 12 - DNA, 87 bp, Artificial Sequence
  Primer 989
gttcagcctgcgcaagtactctggtggcggtggctcgggcggaggtgggtcgggtggcggcggatccgacgtgaagcttgtggagtc

SEQ ID NO: 13 - DNA, 69 bp, Artificial Sequence
  Primer 990
gtccttactttaaaaggtgtccagtgtaagcggctgtttaaggagctcaagttcagcctgcgcaagtac

SEQ ID NO: 14 - DNA, 65 bp, Artificial Sequence
  Primer 991
ggatatccaccatggacttcgggttgagcttggttttccttgtccttactttaaaaggtgtccag

SEQ ID NO: 15 - DNA, 39 bp, Artificial Sequence
  Primer 452
tgggtcgacwgatggggstgttgtgctagctgaggagac

(19) United States
(12) Patent Application Publication
(10) Pub. No.: US 2004/0052814 A1
(43) Pub. Date: Mar. 18, 2004

(54) Fusion proteins for targeted delivery of antimicrobial peptides

(76) Inventors: Wenyuan Shi, Los Angeles, CA (US); Sherie L. Morrison, Los Angeles, CA (US); Kham Trinh, Alhambra, CA (US); Letitia Wims, Culver City, CA (US); Li Chen, Los Angeles, CA (US); Maxwell H. Anderson, Seattle, WA (US)

Correspondence Address: GRAY CARY WARE & FREIDENRICH LLP, 153 TOWNSEND, SUITE 800, SAN FRANCISCO, CA, 94107, US

(21) Appl. No.: 09/910,358

(22) Filed: Jul. 19, 2001

Related U.S. Application Data
(63) Continuation-in-part of application No. 09/378,577, filed on Aug. 20, 1999.
(60) Provisional application No. 60/102,179, filed on Sep. 28, 1998.

(51) Int. Cl. A61K 39/02; A61K 39/002; C07K 14/44; C07K 14/195; C07K 14/39; C07K 14/375

(52) U.S. Cl. 424/190.1; 424/191.1; 530/350

(57) ABSTRACT

Microbial infection may be treated by administration of a fusion protein comprising one or more recognition sequences and at least one antimicrobial peptide. In preferred embodiments, a linker peptide connects the recognition sequence and one or more antimicrobial peptides. The recognition sequence may be an immunoglobulin molecule, or fragment thereof, that specifically binds to a target antigen present on a pathogen. The recognition sequence may also be a non-immunological polypeptide, providing that the polypeptide binds specifically to a particular ligand. In presently preferred embodiments the recognition sequence is monoclonal antibody that binds specifically to S. mutans and the antimicrobial peptides are derivatives of histatin.

This application is a continuation-in-part of U. S. patent application Ser. No. 09/378,577 filed Aug. 20, 1999.

### FIELD OF THE INVENTION

This invention relates to targeted delivery of antimicrobial peptides by fusion proteins. More specifically, this invention relates to a fusion protein comprising a recognition sequence and an antimicrobial peptide. The fusion proteins of the present invention are useful for specifically and selectively destroying or inhibiting the growth of microbial organisms that are associated with parasitic infestations as well as bacterial, ricketsial, protozoan and fungal infections, especially in a complex multi-species environment.

### BACKGROUND OF THE INVENTION

The Centers for Disease Control estimates that half of the more than 100 million annual prescriptions of antibiotics are unnecessary. As a result, microbes have, in many cases, adapted and are resistant to antibiotics due to constant exposure and improper use of the drugs. It is estimated that the annual cost of treating drug resistant infections in the United States is approximately $5 billion. This continued emergence of anti-microbial-resistant bacteria, fungi, yeast and parasites has encouraged efforts to develop other agents capable of killing pathogenic microbes.

Recent research has revealed a class of naturally occuring antimicrobial peptides in humans, other mammals, insects and other organisms. Anti-microbial peptides are usually expressed by various cells in the body including neutrophils and epithelial cells. In mammals, including man, antimicrobial peptides are found on the surface of the tongue, trachea, and upper intestine. Naturally occurring anti-microbial peptides are generally amphipathic molecules that contain fewer than 100 amino acids. Many of these peptides generally have a net positive charge (i.e., cationic) and most form helical structures. Again, speaking generally, the peptides' antimicrobial efficacy is in their ability to penetrate and disrupt the microbial membranes, thereby killing the microbe or inhibiting its growth.

One example of an anti-microbial peptide is histatin. Histatin, and related derivative peptides, posses antifungal and antibacterial activity against a variety of organisms, including Streptococcus mutans. MacKay, B. J. et al., Infect. Immun. 44:695-701 (1984); Xu, et al., J. Dent. Res. 69:239 (1990). S. mutans is believed to be the principal cause of dental caries (tooth decay) in man.

A negative aspect of treatment with antibiotics or anti-microbial peptides is their ability to kill or inhibit the growth of a broad spectrum of organisms. The human body is home to perhaps millions of different bacteria, many of which are vital for optimum health. Overuse of antibiotics can seriously disrupt the normal ecology of the body and render humans more susceptible to bacterial, yeast, viral, and parasitic infection. This effect is also seen with administration of anti-microbial peptides. For example, while histatin has been shown to kill the bacterium primarily responsible for dental caries, general administration of histatin can actually stimulate the growth of oral yeast and other bacteria, such as Actinomyces sp. Accordingly, histatin is not useful by itself for prevention of dental disease.

Another disadvantage of administration of antimicrobial peptides is their ability to damage host cells at higher concentrations since these positively charged peptides can also penetrate and disrupt eukaryotic cell membranes.

Previous efforts to target delivery of pharmaceutically active agents relied principally on non-specific chemical reactions between a pharmaceutically active agent, and a targeting component. For example Shih et al. U. S. Pat. No. 5,057,313 refers to targeting delivery of drugs, toxins and chelators to specific sites in an organism by loading a therapeutic or diagnostic component onto a polymeric carrier, followed by conjugation of the carrier to a targeting antibody. Hansen, U. S. Pat. No. 5,851,527 claims a similar invention. A drawback to this approach is that the non-specific linkage of the pharmaceutical reagents to unknown sites on the antibody molecule used for targeting may interfere with delivery of the therapeutic agents. See Rodwell et al., U. S. Pat. No. 4,671,958. Moreover, chemical modification of a targeting antibody by the nonspecific reactions during conjugation may substantively alter the antibody itself, thereby affecting its binding to targets. Chemical linkage is very inefficient, and the result is non-uniform, making the technique very difficult to use in practice.

More recently, there have been a number of reports of the use of recombinant techniques to produce fusion proteins for the treatment of disease. See Penichet and Morrison, J. Immunological Methods, 248:91-101 (2001) for review. Penichet et al. discuss efforts to treat malignant disease using a genetically engineered protein construct including an immunological component that binds specifically to tumor cells and a cytokine capable of eliciting significant antitumor activity. See, e.g. Pastan et al. U.S. Pat. No. 5,981,726, and Fell, Jr. et al., U.S. Pat. No. 5,645,835.

However, to date there have not been any reports of directing antimicrobial agents to affected regions of humans or animals using target-specific molecules.

### SUMMARY OF THE INVENTION

Microbial infection may be treated by administration of a fusion protein comprising one or more recognition sequences and at least one antimicrobial peptide. In preferred embodiments, a linker peptide connects the recognition sequence and one or more antimicrobial peptides. The recognition sequence may be an immunoglobulin molecule, or fragment thereof, that specifically binds to a target antigen present on a pathogen. The recognition sequence may also be a non-immunological polypeptide, providing that the polypeptide binds specifically to a particular ligand. In presently preferred embodiments the recognition sequence is monoclonal antibody that binds specifically to S. mutans and the antimicrobial peptides are derivatives of histatin.

### BRIEF DESCRIPTION OF THE DRAWINGS

The present invention is now described, by way of illustration only, in the following examples which refer to the accompanying FIGS. 1-4, in which:

FIG. 1 is a schematic diagram of the sequential PCR reactions used to assemble the heavy chain portion of the antibody-based fusion protein.

FIG. 2 shows the DNA sequences (SEQ ID NOS: 9-15) of the primers used in the sequential PCR reactions in embodiments of the present invention.

FIG. 3 shows the DNA sequence (SEQ ID NO: 1) encoding the anti-microbial peptide, histatin 5, the linker peptide, and the variable region of the heavy chain derived from the SWLA3 monoclonal antibody together with the predicted amino acid sequence (SEQ ID NO: 4) of an embodiment of the present invention.

FIG. 4 shows the DNA sequence (SEQ ID NO: 5) encoding the anti-microbial peptide, dhvar 1, the linker peptide, and the variable region of the heavy chain derived from the SWLA3 monoclonal antibody together with the predicted amino acid sequence (SEQ ID NO: 8).

### DETAILED DESCRIPTION OF THE INVENTION

1. Structure of the Fusion Proteins

In various embodiments of the invention, the fusion protein for targeted delivery of antimicrobial peptides comprises at least one recognition sequence that specifically recognizes and binds to a microbe specific antigen. In a presently preferred embodiment of the invention, recognition sequences are derived from SWLA3, a monoclonal antibody that specifically binds to an antigen present on S. mutans. The fusion protein of the present invention further comprises an anti-microbial peptide that is toxic to one or more selected microbes. Preferably, the fusion protein also includes one or more linker peptides connecting the recognition sequences and antimicrobial peptide sequences.

The fusion protein of the present invention includes a recognition sequence that provides for specific targeting. Specific targeting provides for greater concentrations of the antimicrobial peptide at sites where antimicrobial therapy is needed. Thus the present invention can be administered at concentrations that will effectively kill or inhibit growth of harmful microbes, which are low enough to spare the beneficial microbes located elsewhere. As shown in the accompanying examples, a fusion protein according to the present invention comprising histatin 5 or dhvar 1 (both histatin derivatives) a glycine/serine linker peptide, and the SWLA3 variable region has specific anti-microbial activity and antigen specificity against S. mutans.

The linker sequence is expected to facilitate the correct conformation of the recognition sequence and antimicrobial peptides. In addition, by providing flexibility, the linker sequence may be expected to allow antimicrobial peptides to act on microbial membranes.

The present invention provides for dimeric immunoglobulin molecules as well as monomeric or multimeric molecules comprising fusion proteins for targeted delivery of antimicrobial peptides. Further, immunoglobulin fragments that contain enough of the variable region structure to allow for specific antigenic binding may be used in the practice of the invention.

In a preferred embodiment, the immunoglobulin portion of the fusion protein is at least a portion of chimeric antibody whose variable region is derived from a monoclonal antibody that specifically binds to a microbe, and whose constant region is capable of engaging the humoral immune effector systems of the animal to be treated. When the mammal to be treated is human, the constant region is preferably of the IgG or IgM isotypes. Preparation and use of such chimeric monoclonsal antibodies is disclosed and claimed in U.S. Pat. No. 6,231,857 and pending U.S. application Ser. Nos. 09/378,577 and 09/881,823. Use of a chimeric antibody for targeting may provide additional antimicrobial efficacy by engaging the host's humoral immune system.

The fusion proteins of the invention include other anti-microbial peptides. Anti-microbial peptides within the scope of the present invention indolicidin, apidaecin, bacteriocin, α-helical clavanin, magainin, cecropin, andropin, histatin, β-pleated sheet bacteriocin dodecapeptide, tachyplesin, protegrin, defensin, β-defensin, α-defensin (Miyasaki and Lehrer, 1998, Intl. J. Antimicrobial Agents 9:270-272); alexomycin (Marshel and Jones, 1999, Diagn. Micrbiol. Infect. Dis. 33:183-184); nisin, ranalexin, buforin (Giacometti et al., 1999, Peptides 30:1266). Derivatives and analogues of such peptides are likewise within the scope of the present invention. The relatively simple structure of bacteriocidal peptides lends itself to designing a peptide with increased anti-microbial activities and decreased host cell toxicity.

More specifically, buforin, nisin and cecropin have antimicrobial effects on Escherichia. coli, Shigella disenteriae, Salmonella typhimurium, Streptococcus pneumoniae, Staphylococcus aureus, and Pseudomonas aeroginosa.

Magainin and ranalexin have antimicrobial effects on the same organsims, and in addition has such effects on Candida albicans, Cryptococcus neoformans, Candida krusei, and Helicobacter pylori.

Protegrin has antimicrobial effects on Neisseria gonorrhoeae, Chlamydia trachomatis and Haempohilus influenzae.

Alexomycin has antimicrobial effects on Camphylobacter jejuni, Moraxella catarrhalis and Haemophilus inflluenzae.

αdefensin and βpleated sheet defensin have antimicrobial effects on Streptococcus pneumoneae.

In additional embodiments, the antibody fusion proteins of the invention may be directed toward antigens associated with other infectious agents including, ricketsia, fungi, protozoa, and parasites.

The present invention offers the advantage of targeted delivery of antimicrobial peptides, which allows for a lower concentration of anti-microbial peptide to be administered thereby substantially decreasing unintended exposure. Thus side effects due to drug toxicity and unintended exposure of non-pathogenic organisms, including bacteria, may be avoided. In addition, the targeted antimicrobial fusion protein is expected to be readily metabolized by the body because it is based on substances that occur naturally in man and other animals.

The antibody fusion proteins may be administered to a patient in need of such treatment in any sterile pharmaceutical carrier that will maintain the solubility and activity of the protein. In particular, the fusion proteins of the present invention may be administered either topically or by injection.

2. Construction of Recombinant Genes Encoding Targeted Antimicrobial Fusion Proteins

The fusion proteins of the presently preferred embodiment comprise (1) a recognition sequence that specifically binds to a microbe, (2) a linker peptide, and (3) an anti-microbial peptide. In the presently preferred embodiment of the invention, the fusion proteins are synthesized by cells transformed as described below. The recombinant genes encoding the fusion proteins of the invention may be constructed using any technique known in the art of molecular biology, including but not limited to the following.

The targeting sequence of the fusion protein may comprise all or part of an immunoglobulin variable region which may, in turn, be comprised of regions encoded by a V gene and/or D gene and/or J gene. Alternatively, the recognition sequence may be any polypeptides that specifically binds to a pathogen. For example the recognition sequence may be glucosyl transferase, which specifically binds to glucans on the surface of certain bacteria.

In preferred embodiments of the invention, the antibody fusion proteins preferably include a peptide linker that joins the anti-microbial peptide and the recognition sequence. The linker is preferably located between the anti-microbial peptide and upstream of the variable region of the heavy chain, when the targeting sequence is an immunoglobulin molecule. The linker may be important in retaining antibody conformation to provide specificity while allowing the anti-microbial peptide to interact with the microbial membrane. The antimicrobial peptide could also be fused to the C terminus of an immunoglobulin protein or portion thereof. It should be understood that any peptide that would provide flexibility between the peptide and the variable region of the antibody would be functionally equivalent. Variable regions from complete or incomplete antibodies, particularly monoclonal antibodies, which recognize specific parasite, ricketsial, bacterial, yeast or fungal antigens expressed on a particular population of microbes may be used in fusion proteins of the invention.

Recombinant nucleic acid molecules that encode the immunoglobulin, linker or anti-microbial peptide may be obtained by any method known in the art. See, e.g. Maniatis et al., 1982, Molecular Cloning; A Laboratory Manual, Cold Spring Harbor Laboratory, Cold Spring Harbor, N.Y. or obtained from publicly available clones.

For example, a population of cells known to actively express the peptide is obtained, and total cellular RNA may be harvested therefrom. The amino acid sequence of the peptide sought may be used to deduce the sequence of a portion of the peptide's nucleic acid so as to design appropriate oligonucleotide primers, or, alternatively, the oligonucleotide primers may be obtained from a known nucleic acid sequence which encodes the peptide. The oligonucleotide fragment may then be used in conjunction with reverse transcriptase to produce cDNA corresponding to peptide-encoding nucleotide sequence. Okayama et al., 1987, Methods Enzymol. 154:3-29. The cDNA can then be cloned, and/or portions of the peptide coding region may then be amplified from this cDNA using polymerase chain reaction and appropriate primer sequences. Saiki et al., 1988, Science 239:487-491. Alternatively, specific nucleic acid sequences encoding the anti-microbial peptides disclosed in this application are available in the references cited above and in the sequence listings presented below.

In preferred embodiments of the invention, a recombinant vector system was created to accommodate sequences encoding the anti-microbial peptide in the correct reading frame with the linker peptide and immunoglobulin. For example, and not by way of limitation, sequential PCR reactions were done to add on portions of the fusion protein described above. In the first reaction the linker peptide was added upstream of the variable region of the heavy chain. The second PCR reaction added the anti-microbial peptide upstream from the peptide. In the final reaction, a signal peptide was added to the 5′ flanking region of the anti-microbial peptide gene to facilitate the secretion of the fused molecule from a cell transformed with the recombinant vector. Once the proper orientation was confirmed, the clone was inserted using standard restriction enzyme techniques into a human IgG1, expression vector.

Nucleic acid sequences encoding the various components of the fusion proteins of the present invention may be joined together using any techniques known in the art, including the use of synthetic linker sequences and restriction enzyme methodologies.

Various techniques are known in the art for transcription of recombinant constructs of the invention. One such technique is incorporating a suitable promoter/enhancer sequence into the expression vector. Promoters which may be used to control the expression of the antibody-based fusion protein include, but are not limited to, the promoter contained in the 3′ long terminal repeat of Rous sarcoma virus (Yamamoto, et al., 1980, Cell 22:787-797), the SV40 early promoter region (Bernoist and Chambon, 1981, Nature 290:304-310), the herpes thymidine kinase promoter (Wagner et al., 1981, Proc. Natl. Acad. Sci. U.S.A. 78:1444-1445), and the regulatory sequences of the metallothionine gene (Brinster et al., 1982, Nature 296:39-42).

3. Expression of Fusion Proteins for Targeted Delivery of Antimicrobial Peptides

The recombinant constructs of the present invention may be introduced into host cells using any method known in the art, including transfection, transformation, microinjection, infection, cell gun, and electroporation. Any host cell type may be utilized provided that the antibody-based fusion protein recombinant nucleic acid sequences can be accurately transcribed into mRNA in that cell type. In specific embodiments of the invention, mouse myeloma cell lines which do not produce immunoglobulin, such as SP2/0 or P3X63.Ag8.653 were cotransfected by electroporation.

In specific embodiments of the invention, the genetically engineered nucleic acid sequence which codes for the signal peptide/anti-microbial peptide/linker peptide/immunoglobulin variable region construct was cloned into an IgG₁, expression vector, which contained the immunoglobulin heavy chain constant region. The light chain variable region was cloned into a human kappa expression vector, which contained the immunoglobulin light chain constant region. In each case, and not by way of limitation, the genetic sequence coding for the variable region of the heavy and light chain of the fusion protein was derived from a hybridoma which produces a monoclonal antibody to S. Mutans (SWLA3). The hybridoma used in the preferred embodiment is deposited with the American Type Culture Collection, HB 12558. U.S. Pat. No. 6,231,857. In one embodiment of the invention, only the variable region of the heavy chain (VH SWLA3) is used in preparing the construct used in the present invention. This sequence was cloned and the various components of the fusion protein were sequentially added using known PCR techniques. Once complete, the entire construct was cloned into an IgG₁, expression vector using known restriction enzyme techniques. The resulting heavy and light chain expression vectors are then cotransfected into a given host cell and the complete antibody-based fusion protein is expressed.

Alternatively, the host cell is a hybridoma derived heavy chain loss variant which expresses only immunoblobulin light chains. Preferably, the hybridoma is derived from a parent that produces monoclonal antibodies specific for the desired antigen. Thus, the derived light chain producing host may be transfected with the recombinant heavy chain expression vector; leading to the expression of an antibody-based fusion protein with the same antigen specificity of the monoclonal antibody produced by the parent hybridoma. In addition, the recombinant nucleic acid constructs of the invention may be used to create non-human transgenic animals capable of producing the targeted antibmicrobial fusion protein of the present disclosure.

To determine whether the antibody-based fusion gene has been successfully incorporated into a given host cell, three general methods are known: (1) expression of inserted sequences (2) DNA-DNA hybridization, and (3) presence or absence of “marker” gene functions. In the first method, recombinant expression vectors can be identified by assaying the foreign gene product expressed by the recombinant. Such assays can be based, for example, on the physical or functional properties of the antibody fusion gene product in bioassay systems as described infra. In the second method, the presence of a foreign gene inserted in an expression vector can be detected by DNA-DNA hybridization using probes comprising sequences that are homologous to the inserted antibody fusion protein gene. In the third method, the recombinant vector/host system can be identified and selected based upon the presence or absence of certain “marker” gene functions which include antibiotic resistance and transformation phenotype caused by the insertion of foreign genes in the vector. For example, if the antibody fusion gene was inserted so as to interrupt the marker gene sequence of the vector, recombinant constructs containing the antibody fusion gene insert can be identified by the absence of the marker gene function. Alternatively, gene additions that confer identifiable marker phenotypes in the transformed cell can be employed. In the present invention, successfully incorporated constructs were discovered with the observation that the cells were resistant to drugs such as histidinol or mycophenolic acid.

Once a particular recombinant DNA molecule is identified and isolated, several methods known in the art may be used to propagate it. Once a suitable host system and growth conditions are established, recombinant expression vectors can be propagated and prepared in quantity. The expression vectors which can be used include, but are not limited to, the following vectors or their derivatives: human or animal viruses such as vaccinia virus, adenovirus or retroviral based vectors; insect viruses such as baculovirus; yeast vectors; bacteriophage vectors (e.g., lambda), and plasmid and cosmid DNA vectors, to name but a few.

4. Analysis of Targeted Antimicrobial Fusion Proteins

The fusion proteins of the present invention may be produced by cells transformed as previously described, and may be collected using any technique known in the art, including, but not limited to, affinity chromatography using target antigen or antibody specific for any portion of the fusion protein including, for example, anti-idiotype antibody. The activity of the fused anti-microbial peptide may be confirmed using biological assays which detect whether the antibody is antigen specific and whether the anti-microbial peptide is effective in destroying the microbe or inhibiting its growth.

In a specific embodiment of the invention, the specificity of the antibody-based fusion protein to S. mutans was measured using both fluorescent microscopy and flow cytometry. The desired targeting of the fused anti-microbial peptide was confirmed by observing that the antibody fusion protein results in the destruction of S. mutans using an anti-microbial peptide concentration that would normally not affect the microbe.

### EXAMPLES

1. Construction and Expression of a Histatin 5 and Dhvar 1/SWLA3 Chimeric Antibody Fusion Protein with Activity Against S. mutans.

a. Construction of an expression vector for an antibody-based fusion protein

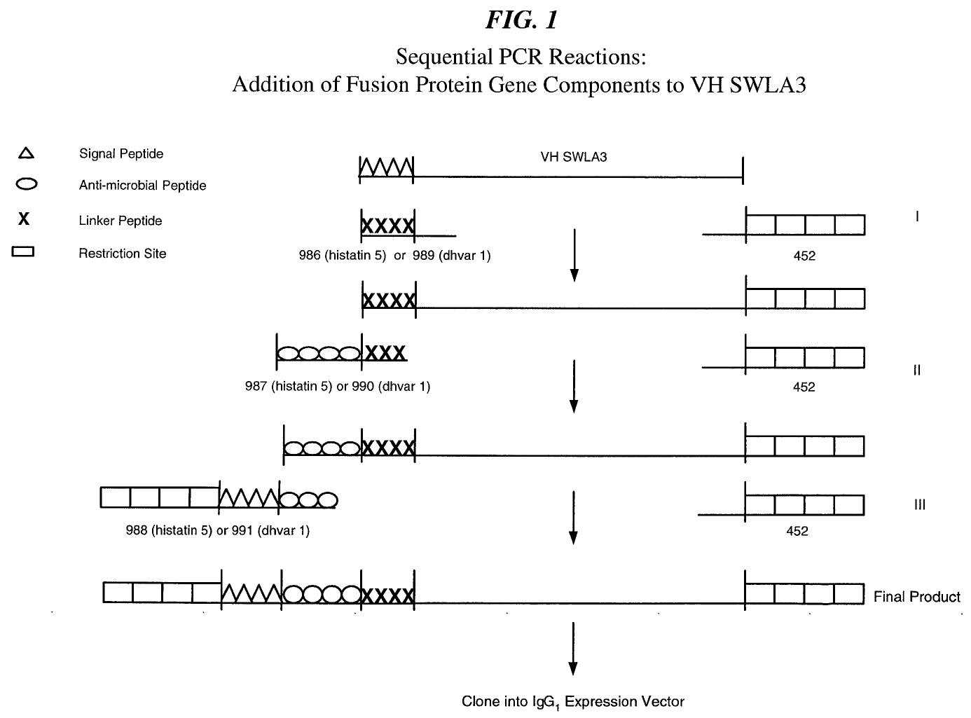

The construct that is ultimately cloned into an IgG₁ expression vector and leads to the expression of the targeted antimicrobial fusion protein was assembled according to the following method (see FIG. 1). The construct was assembled using sequential PCR and restriction enzymes techniques. The recognition sequence of the of the fusion protein was derived from heavy chain sequences of SWLA3, produced by hybridoma ATCC HB 12558. See Shi, U.S. Pat. No. 6,231,857, the disclosure of which is incorporated by reference, and U.S. Ser. Nos. 09/378,577 and 09/881,823. Sequences that code on expression for Histatin 5 or dhvar1 were inserted upstream of the variable region of the heavy chain of SWLA3. The amino acid sequences used for histatin 5 and dhvar 1 are listed below:

| | | |
|---|---|---|
| Histatin | DSHAKRHHGY KRKFHEKHHS HRGY | (SEQ ID NO: 2) |
| 5 | | |
| Dhvar 1 | KRLFKELKFS LRKY. | (SEQ ID NO: 6) |

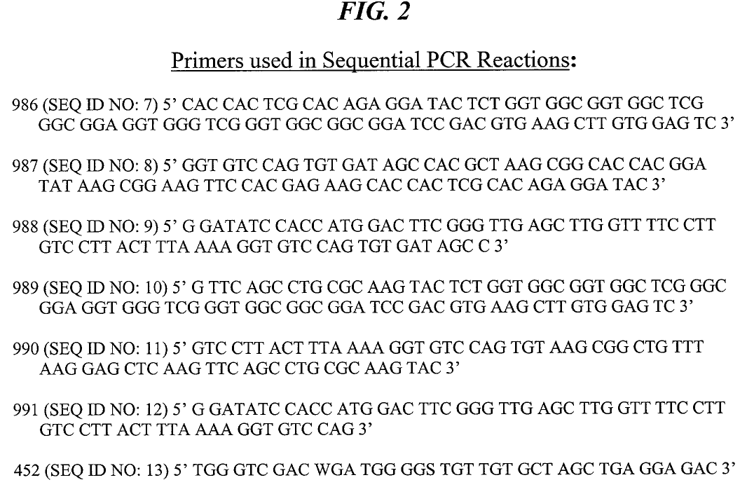

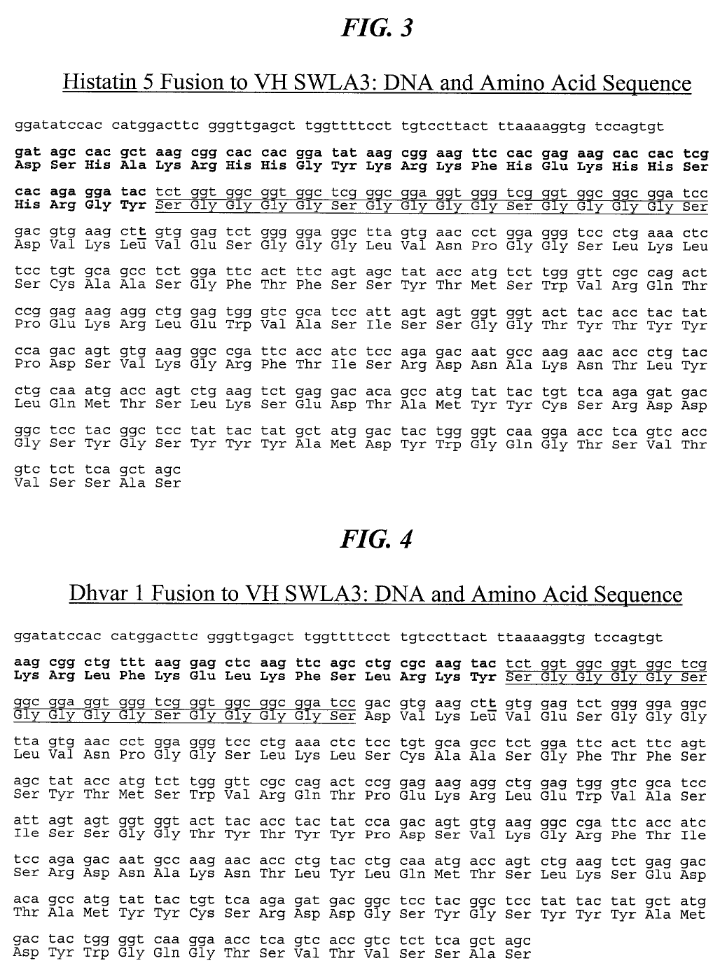

The source signal peptide was added upstream of the histatin 5 or dhvar1, and a glycine/serine linker (SEQ ID NOS: 3 and 7) was added to separate the fusion protein from the variable region of the heavy chain (VH) of the antibody. See FIG. 3 for the nucleic acid and predicted amino acid sequence for the histatin 5/SWLA3 VH and FIG. 4 for the respective dhvar 1/SWLA3 VH sequences. Sequential PCR reactions were used to complete the construct according to the following method (see FIG. 2 for the nucleic acid sequence of the primers used):

1. In the first PCR reaction a plasmid carrying the VH of SWLA3 was used as the template with primer sets 986+452 (histatin 5) or 989+452 (dhvar1). This reaction replaced the signal peptide in the original gene with the linker peptide at the 5′ end of the VH and inserted a restriction site at the 3′ end. The products of this reaction were isolated and used a template in the second PCR reaction.

2. Using primer sets 987+452 (histatin 5) or 990+452 (dhvar1) in the second PCR reaction added the anti-microbial peptide upstream from the linker peptide. The restriction site at the 3′ end was maintained. The products from this reaction were isolated and used as the template in the third PCR reaction.

3. With primer sets 988+452 (histatin 5) or 991+452 (dhvar1) a signal peptide and restriction site were added upstream from the anti-microbial peptide. The restriction site at the 3′ end was maintained. Products from the third PCR were isolated.

4. Isolated products from the third PCR reaction were then cloned into Invitrogen's PCR2.1 vector via TOPO Cloning Kit and sequenced.

5. After the sequences of the two clones were confirmed, the inserts were moved into the IgG₁, PCR expression vector (pAH 4604) as an NheI/EcoRV fragment.

6. The final expression vectors for the histatin 5 and dhvar 1 antibody fusion proteins were named pAH 5993 and pAH 5994 respectively.

PCR conditions used were:

1. Denature @ 94° C. for 40 sec.

2. Anneal @ 60° C. for 40 sec.

3. Extend @ 72° C. for 40 sec.

4. Amplify for 30 cycles

5. Final Extension at 72° C. for 10 min.

FIG. 3 shows the nucleic acid sequence encoding the histatin 5 fusion to VH SWLA3 and predicted amino sequence (SEQ ID NOS: 1 and 4) and FIG. 4 which shows the nucleic acid sequence encoding the dhvar1 fusion to VH SWLA3 and predicted amino sequence (SEQ ID NOS: 5 and 8). In the figures, the bold sequences represent the corresponding anti-microbial peptides, the underlined sequences represent the glycine/serine linker, and the single bolded underlined base in each sequence represents a silent point mutation. In the original sequence disclosed in Shi et al. Shi et al. U.S. Ser. No. 09/881,823, the base is guanine.

The variable region of the light chain (VL) from SWLA3 was cloned into a human kappa expression vector named 5940 pAG according to the method described in Shi et al. U.S. Ser. No. 09/881,823. Briefly,

(i) DNA was prepared from the expression vectors and from the plasmid containing the correct VL. See Current Protocols in Immunology, Section 2.12.1 (1994) for detailed information about the vectors that express the light and heavy chain constant regions.

(ii) The expression vector was digested with the appropriate restriction enzyme. The digests were then electrophoresed on an agarose gel to isolate the appropriate sized fragment.

(iii) The plasmid containing the cloned VL region was also digested and the appropriate DNA fragment containing the VL region was isolated from an agarose gel.

(iv) The VL region and expression vector were then mixed together, T4 DNA ligase was added and the reaction mixture was incubated at 16° C. over night.

(v) Competent cells were transfected with the VL ligation mixture and the clones expressing the correct ligation sequence were selected. Restriction mapping was used to confirm the correct structure.

b. Transfecting Eukaryotic Cells

10 micrograms of DNA from each expression vector, pAH 5993 (histatin 5) or pAH 5994 (dhvar 1) and 5940 pAG, was linearized by BSPC1 (Stratagene, PvuI isoschizomer) digestion and 1×10⁷ myeloma cells (SP2/0 or P3X63.Ag8.653) were cotransfected by electroporation. Prior to transfection the cells were washed with cold PBS, then resuspended in 0.9 ml of the same cold buffer and placed in a 0.4 cm electrode gap electroporation cuvette. 960 microF and 200 V was used for electroporation. The shocked cells were then incubated on ice in IMDM medium (Gibco, N.Y.) with 10% calf serum.

The transfected cells were plated into 96 well plates at a concentration of 10000 cells/well. Selective medium including selective drugs such as histidinol or mycophenolic acid were used to select the cells which contain expression vectors. After 12 days, the supernatants from growing clones were tested for antibody production.

2. Analyses of Recombinant Antibody-Based Fusion Proteins

ELISA assay was used to identify transfectomas that secrete the fusion IgG antibodies. 100 μl of 5 μg/ml goat anti-human IgG was added to each well of a 96-well ELISA plate and incubated overnight. The plate was washed several times with PBS and blocked with 3% BSA. Supernatants from above growing clones were added to the plate for 2 hours at room temperature to assay for their reactivity with goat anti-human Ig antibody. Plates were then washed and anti-human kappa antibody labeled with alkaline phosphatase diluted 1:10⁴ in 1% BSA was added for 1 hour at 37° C. Plates were washed with PBS and p-NPP in diethanolamine buffer (9.6% diethanolamine, 0.24 mM MgCl₂, pH 9.8) was added. Color development at OD₄₀₅ was indicative of cells producing H₂L_{2.}

For the supernatants that produce IgG constant regions, their reactivity with S. mutans was tested as described in Shi et al., Hybridoma 17:365-371 (1998). Briefly, bacteria strains listed in Table 1 were grown in various media suggested by the American Type Culture Collection. The anaerobic bacteria were grown in an atmosphere of 80% N_{2,} 10% CO₂, and 10% H₂ at 37° C. The specificity of antibodies to various oral bacteria was assayed with ELISA assays. Bacteria were diluted in PBS to OD₆₀₀=0.5, and added to duplicate wells (100 μl) in 96 well PVC ELISA plates preincubated for 4 h with 100 μl of 0.02 mg/ml Poly-L-lysine. These antigen-coated plates were incubated overnight at 4° C. in a moist box then washed 3 times with PBS and blocked with 0.5% fetal calf serum in PBS and stored at 4° C. 100 μl of chimeric antibodies at 50 μg/ml were added to the appropriate wells of the antigen plates, incubated for 1 h at RT, washed 3 times with PBS-0.05% Tween 20, and bound antibody detected by the addition of polyvalent goat-anti-human IgG antibody conjugated with alkaline phosphatase diluted 1:10³ with PBS-1% fetal calf serum. After the addition of the substrate, 1 mg/ml p-nitrophenyl phosphate in carbonate buffer (15 mM Na₂CO_{3,} 35 mM NaH₂CO_{3,} 10 mM MgCl₂ pH 9.6), the color development after 15 min was measured in a EIA reader at 405 nm. “+”means OD405>1.0; “−” means OD405<0.05. The negative control is <0.05. The results are given in Table 1.

TABLE 1
Reactivity of Antibody Fusion Proteins to Various Oral Bacterial Strains

| | | Hitstatin | Dhvar |
|---|---|---|---|
| | | 5/SWLA3 | 1/SWLA3 |
| | | Fusion | Fusion |
| Oral Bacteria | Strains | Antibodies | Antibodies |
| S. mutans | AATCC25175 | + | + |
| | LM7 | + | + |
| | OMZ175 | + | + |
| S. Mitis | ATCC49456 | − | − |
| S. rattus | ATCC19645 | − | − |
| S. sanguis | ATCC49295 | − | − |
| S sobrinus | ATCC6715-B | − | − |
| S. sobrinus | ATCC33478 | − | − |
| L. acidophilus | ATCC4356 | − | − |
| L. casei | ATCC11578 | − | − |
| L. plantarum | ATCC14917 | − | − |
| L. salivarius | ATCC11742 | − | − |
| A. actinomycetemcomitans | ATCC33384 | − | − |
| A. naeslundi | ATCC12104 | − | − |
| A. viscosus | ATCC19246 | − | − |
| Fusobacterium nucleatum | ATCC25586 | − | − |
| Porphyromonas gingivalis | ATCC33277 | − | − |

The fusion proteins showed both specificity and antimicrobial efficacy against S. mutans. Like the monclonal antibodies from which they are derived, the fusion proteins bind specifically to S. mutans. They also have anti-bacterial efficacy against the bacteria, but are effective at a much lower concentration than histatin 5 alone. This observation suggests that the recognition sequence is responsible for specific binding between the fusion protein and S. mutans, which locally enhances the concentration of histatin 5 at the bacterial cell surface. At the concentration at which the fusion protein showed antibacterial efficacy, the fusion proteins showed no inhibitory effect on other bacteria or host cells. Accordingly, these results suggest that the basic design described herein may be useful for generating antibody-based fusion proteins for treatment of other infections and infestations.

While various embodiments are disclosed in this application, it is apparent that the invention can be altered to provide other embodiments that utilize the composition and process of the invention. Therefore it will be appreciated that the scope of the invention is to be defined by the claims appended hereto rather than by the specific embodiments and examples that have been disclosed for the purposes of illustrating and explaining the invention.

What is claimed is:

1. A fusion protein for the targeted delivery of antimicrobial peptides comprising a recognition sequence that specifically binds to a microbial organism, and an anti-microbial peptide.

2. The fusion protein for the targeted delivery of antimicrobial peptides of claim 1, further comprising a linker peptide.

3. The fusion protein for the targeted delivery of antimicrobial peptides of claim 1, wherein the microbe is selected from the group consisting of bacteria, ricketsia, fungi, yeasts, protozoa and parasites.

4. The fusion protein for the targeted delivery of antimicrobial peptides of claim 1, wherein the antimicrobial peptide is selected from a group consisting of histatin, defensin, magainin, cecropin, cathelicidin, buforin, gaegurin, indolicidin, tachyplesin, andropin, bactenecin, protegrin, apidaecin, bacteriocin, clavanin, alexomycin, nisin, and ranalexin and deriviatives thereof.

5. The fusion protein for the targeted delivery of antimicrobial peptides of claim 1, wherein the microbe is a cariogenic organism.

6. The fusion protein for the targeted delivery of antimicrobial peptides of claim 5, wherein the microbe is Streptococcus mutans.

7. The fusion protein for the targeted delivery of antimicrobial peptides of claim 6, wherein the antimicrobial peptide is histatin 5, which is coded for by the nucleic acid sequence designated SEQ ID NO: 1.

8. The fusion protein for the targeted delivery of antimicrobial peptides of claim 6, wherein the antimicrobial peptide is, dhvar1, which is coded for by the nucleic acid sequence designated SEQ ID NO: 5.

9. The fusion protein for the targeted delivery of antimicrobial peptides of claim 6, wherein the recognition sequence is at least a portion of a variable region of an immunoglobulin that specifically binds to S. mutans, and the antimicrobial peptide is selected from the group consisting of histatin 5, having the amino acid sequence designated SEQ ID NO: 4, and dhvar1, having the amino acid sequence designated SEQ. ID NO: 8.

10. A method of treating microbial infection comprising exposing the microbe to a fusion protein comprising a recognition sequence that specifically binds to the microbe and an antimicrobial peptide

11. The method of claim 10, wherein the microbe is Streptococcus mutans and the anti microbial peptide is selected from a group consisting histatin 5 and dhvar 1.

12. The method of claim 10, wherein the anti-microbial peptide is buforin and the microbe is selected from a group consisting Escherichia coli, Shigella dysenteriae, Salmonella typhimurium, Streptococcus pneumoniae, Staphylococcus aureus, and Pseudomonas aeruginosa.

13. The method of claim 10, wherein the anti-microbial peptide is a cecropin and the microbe is selected from a group consisting Escherichia coli, Shigella dysenteriae, Salmonella typhimurium, Streptococcus pneumoniae, Staphylococcus aureus, and Pseudomonas aeruginosa.

14. The method of claim 10, wherein the anti-microbial peptide is an indolicidin and the microbe is selected from a group consisting of Escherichia coli, Shigella dysenteriae, Salmonella typhimurium, Streptococcus pneumoniae, Staphylococcus aureus, and Pseudomonas aeruginosa.

15. The method of claim 10, wherein the anti-microbial peptide is a magainin and the microbe is selected from a group consisting Escherichia colt, Shigella dysenteriae, Salmonella typhimurium, Streptococcus pneumoniae, Staphylococcus aureus, and Pseudomonas aeruginosa, Candida albicans, Cryptococcus neoformans, Candida krusei, Helicobacter pylori, and herpes simplex virus.

16. The method of claim 10, wherein the anti-microbial peptide is nisin and the microbe is selected from a group consisting Escherichia coli, Shigella dysenteriae, Salmonella typhimurium, Streptococcus pneumoniae, Staphylococcus aureus, and Pseudomonas aeruginosa.

17. The method of claim 10, wherein the anti-microbial peptide is ranalexin peptide and the microbe is selected from a group consisting Escherichia coli, Shigella dysenteriae, Salmonella typhimurium, Streptococcus pneumoniae, Staphylococcus aureus, Pseudomonas aeruginosa, Candida albicans, Cryptococcus neoformans, Candida krusei, and Helicobacter pylori.

18. The method of claim 10, wherein the anti-microbial peptide is protegrin and the microbe is selected from a group consisting Neisseria gonorrhoeae, Chlamydia trachomatis, and Haemophilius ducreyi.

19. The method of claim 10, wherein the anti-microbial peptide is alexomycin and the microbe is selected from a group consisting Camphylobacter jejuni, Moraxella catarrhalis, and Haemophilius influenzae.

20. The method of claim 10, wherein the anti-microbial peptide is selected from the group consisting of defensin, αdefensin and βpleated sheet defensin and the microbe is Streptococcus pneumoniae.

21. The method of claim 11 wherein the recognition sequence is at least a portion of a variable region of an immunoglobulin selected from the group consisting of SWLA1, SWLA2 and SWLA3.

22. The fusion protein of claim 1 wherein the recognition sequence is a polypeptide and its target is a ligand.
